(19) United States
(12) Patent Application Publication
(10) Pub. No.: US 2024/0182986 A1
(43) Pub. Date: Jun. 6, 2024

(54) ANASTASIS BIOSENSOR CASPASE TRACKER

(71) Applicant: THE JOHNS HOPKINS UNIVERSITY, Baltimore, MD (US)

(72) Inventors: Ho Lam Hogan Tang, Baltimore, MD (US); Ho Man Holly Tang, Baltimore, MD (US)

(21) Appl. No.: 18/312,196

(22) Filed: May 4, 2023

Related U.S. Application Data
(63) Continuation of application No. 16/763,849, filed on May 13, 2020, now Pat. No. 11,649,509.
(60) Provisional application No. 62/587,201, filed on Nov. 16, 2017.

(51) Int. Cl. C12Q 1/6897; C07K 14/00; C12Q 1/34

(52) U.S. Cl. CPC C12Q 1/6897; C07K 14/00; C12Q 1/34; G01N 2333/96413; G01N 2510/00

(57) ABSTRACT

The present invention relates to the field of anastasis, i.e., the process of reversal of apoptosis. More specifically, the present invention provides methods and compositions useful for studying anastasis. In one embodiment, a tracking construct of the present invention comprises Lyn11-NES-ERT2-DEVD-rtTA-3×FLAG-DEVD-ERT2-NES. In another embodiment, a construct comprises Lyn11-NES-DEVD-rtTA-3×FLAG. In a further embodiment, a construct comprises ERT2-DEVD-rtTA-3×FLAG-DEVD-ERT2.

### CROSS-REFERENCE TO RELATED APPLICATIONS

This application claims the benefit of U.S. Provisional Application No. 62/587,201, filed Nov. 16, 2017, which is incorporated herein by reference in its entirety.

### FIELD OF THE INVENTION

The present invention relates to the field of anastasis, i.e., the process of reversal of apoptosis. More specifically, the present invention provides methods and compositions useful for studying anastasis.

### REFERENCE TO AN ELECTRONIC SEQUENCE LISTING

The text of the computer readable sequence listing filed herewith, titled “P15014-04”, created Feb. 26, 2024, having a file size of 91,143 bytes, is hereby incorporated by reference in its entirety.

### BACKGROUND OF THE INVENTION

Programmed cell death such as apoptosis plays essential role in embryonic development and normal hemostasis by eliminating unwanted, injured, or dangerous cells in multicellular organisms. See Fuchs, Y. & Steller, H., 147 CELL 742-58 (2011); Jacobson et al, 88 CELL 347-54 (1997); and Kerr et al., 26 BR. J. CANCER 239-57 (1972). The loss of balance between cell death and survival is fatal consequences such as cancer, heart failure, autoimmunity, and degeneration. See Hanahan, D. & Weinberg, R. A., 144 CELL 646-74 (2011); Nagata, S., 1209 ANN. N. Y. ACAD. SCI. 10-16 (2010); Mattson, M. P., 1 NAT. REV. MOL. CELL. BIOL. 120-29 (2000); and Narula et al., 3335 N. ENGL. J. MED. 1182-89 (1996). Activation of executioner caspases has traditionally been considered as the “point of no return” in apoptosis (Riedl, S. J. & Shi, Y., 5 NAT. REV. MOL. CELL. BIOL. 897-907 (2004); Green, D. & Kroemer, G., 8 TRENDS CELL. BIOL. 267-71 (1998)), as it triggers rapid and massive cellular demolition (Chipuk et al., 37 MOL. CELL. 299-310 (2010); Taylor et al., 9 NAT. REV. MOL. CELL. BIOL. 231-41 (2008); Luthi, A. U. & Martin, S. J., 14 CELL DEATH DIFFER. 641-50 (2007); and Takemoto et al., 160 J. CELL. BIOL. 235-43 (2003)). Challenging this general dogma, we demonstrated that dying primary cells and cancer cells can recovery no only after caspase activation, but also important cell death events including plasma membrane blebbing, cell shrinkage, mitochondrial fragmentation, release of mitochondrial cytochrome c into the cytosol, nuclear and chromatin condensation, DNA damage, nuclear fragmentation, cell surface exposure of phosphatidylserine (PS), and formation of apoptotic bodies. See Tang et al., 43 F1000RES. 43 (2017); Tang et al., 96 J. VIS. EXP. 51964 (2015); Tang et al., 5 SCI. REP. 9015 (2015); Tang et al., 23 MOL. BIOL. CELL 2240-52 (2012); and Tang et al., 100 BR. J. CANCER 118-22 (2009). We propose that anastasis is an intrinsic cell recovery phenomenon, as dying cells can recover after removal of cell death stimuli. Our observation on reversal of cell death process is further supported by independent studies that also reveal recovery of cell after phosphatidylserine externalization (Kenis et al, 51 J. NUCL. MED. 259-67 (2010); Geske et al., 8 CELL DEATH DIFFER. 182-91 (2001); and Hammill et al., 251 EXP. CELL. RES. 16-21 (1999)), cytochrome c release (Ichim et al., 57 MOL. CELL 860-72 (2015)), activation of mixed lineage kinase-like (MLKL) and cell shrinkage (Gong et al., 169 CELL 286-300 E16 (2017)). We coined the term “Anastasis” (Aναστáσηζ) (Tang et al. (2012)), which means “rising to life” in Greek, to describe this unexpected cell recovery phenomenon.

The discovery of anastasis leads to paradigm-shifting physiological, pathological, and therapeutic implications. Anastasis could represent a previously unknown cytoprotective mechanism to rescue and preserve important cells and tissues that are difficult to be replaced (Tang et al. (2012)), thereby underlying the observation on heart failure reversal by ventricular unloading with left ventricular assist devices (LVADs) (Drakos et al., 126 CIRCULATION 230-41 (2012); Narula et al., 3 NAT. CLIN. PRACT. CARDIOVASC. MED. 681-88 (2006)), recovery of photoreceptor cells after transient exposure of excessive light (Gordon et al., 43 INVEST. OPHTHALMOL. VIS. SCI. 3511-21 (2002); Milligan et al., 139 J. CELL. BIOL. 351-63 (1997); McKechnie, N. M. & Foulds, W. S., 212 ALBRECHT VON GRAEFES ARCH. KLIN. EXP. OPHTHALMOL. 271-83 (1980)), repair of neurons after brain injury (Blennow et al., 2 NAT. REV. DIS. PRIMERS 16084 (2016)). If so promoting anastasis could enhance tissue recovery. Anastasis could be an unexpected escape tactic used by cancer cells to survive cell-death-inducing cancer therapy, causing cancer recurrence (Tang et al. (2009)). Therefore, suppressing anastasis in dying cancer cells during and after cancer treatment could be a novel therapeutic strategy to cure cancers by inhibiting cancer relapse. Interestingly, we found that some recovered cells acquired permanent genetic alterations and underwent oncogenic transformation, possibly by rescuing the cells that had experienced DNA damage during apoptosis (Tang et al. (2017); Tang et al. (2015); Tang et al. (2012)). Thus, anastasis could be a tumorigenic mechanism as it rescues DNA damaged cells, underlying the observations that repeated tissue injury increases the risk of cancers in a variety of tissues, such as chronic thermal injury in the esophagus induced by the consumption of very hot beverages (Loomis et al., 17 LANCET ONCOL. 877-78 (2016); Islami et al., 338 BMJ b929 (2009); Castellsague et al., 88 INT. J. CANCER 658-64 (2000)), liver damage due to alcoholism (Boffetta, P. & Hashibe, M., 7 LANCET ONCOL. 149-56 (2006); McKillop, I. H. & Schrum, L. W., 35 ALCOHOL 195-203 (2005)), tumor evolution after genotoxic cancer therapy (Wagle et al., 29 J. CLIN. ONCOL. 3085-96 (2011); Demedts et al., 35 EUR. RESPIR. J. 202-15 (2010); Davis, A. J. & Tannock, J. F., 1 LANCET ONCOL. 86-93 (2000)) and development of second (new) cancers from normal tissues during the intervals between cycles of cancer therapy (Chaturvedi et al., 99 J. NATL. CANCER INST. 1634-43 (2007); Travis et al., 97 J. NATL. CANCER INST. 1354-65 (2005); Smith et al., 21 J. CLIN. ONCOL. 1195-1204 (2003)). If true targeting anastasis could prevent or arrest cancer development and progression.

To harness the discovery of anastasis to develop revolutionary new therapies, it is essential to study the cause and consequence of anastasis in live animals. However, it is technically challenging to identify anastatic cells in vivo, because the cells that recovered from cell death process appear morphologically indistinguishable from normal healthy cells, and there is no biomarker of anastasis (Tang et al. (2017); Tang et al. (2015); Tang et al. (2012)). To address these problems, we recently developed a new in vivo caspase biosensor designated “CaspaseTracker” (Tang et al., 5 SCI. REP. 9015 (2015)), to identify and track cells that survive apoptosis after caspase activation (Ding et al., 5 ELIFE (2016); Tang et al., 5 SCI. REP. 9015 (2015)), the hallmark of apoptosis (Taylor et al. (2008); Riedl et al. (2004)). Different from the “real-time” caspase biosensors such as SCAT (Takemoto et al., 104 PROC. NATL. ACAD. SCI. U.S.A. 13367-72 (2007); Takemoto et al. (2003)), Apoliner (Bardet et al., 105 PROC. NATL. ACAD. SCI. U.S.A. 13901-95 (2008)), CA-GFP (Nicholls et al., 286 J. BIOL. CHEM. 24977-86 (2011)), ApoAlert (Tang et al. (2012); Golbs et al., 21 CEREB. CORTEX 1192-1201 (2011)), C3AIs (Zhang et al., 4 NAT. COMMUN. 2157 (2013)) and iCasper (To et al., 112 PROC. NATL. ACAD. SCI. U.S.A. 3338-43 (2015)) that detect the on-going caspase activity, the CaspaseTracker biosensor can further permanently label the cells that have ever experienced caspase activity. Therefore, the CaspaseTracker biosensor provides long term tracking of anastasis after reversal of caspase-mediated cell death process in vivo.

### BRIEF DESCRIPTION OF THE FIGURES

FIG. 1A-1D. Recovery of HeLa cells after cell death induction. FIG. 1A—Schematic diagram of the approach to induce cell death and subsequently allow dying cells to recover after removal of cell death inducer. FIG. 1B—Time-lapse live cell DIC microscopy of healthy HeLa cells (i), the same group of cells that were treated with 1 μM staurosporine (ii), and then washed and further inducible with fresh culture medium (iii-vi). White arrow indicates the dividing cell. FIG. 1C—Schematic diagram of caspase biosensor fusion protein NES-DEVD-YFP-NLS, for the subcellular localization of YFP during apoptosis and after anastasis. FIG. 1D—Time-lapse live cell confocal microscopy of HeLa cell expressing caspase biosensor fusion protein NES-DEVD-YFP-NLS before (i), during (ii-iii) and after (iv-viii) exposure to 3.7% ethanol in cell culture medium. Confocal images of the caspase biosensor (green, top panel); Merged images of Hoechst-stained nucleus (blue) and mitochondria (red) (middle panel), and further merged with DIC images (bottom panel). White arrows indicate the caspase activated, nuclear localized YFP biosensor signal.

FIG. 2A-2E. Mammalian CaspaseTracker biosensor system. FIG. 2A—Schematic diagram of the mammalian CaspaseTracker rtTA biosensor system. FIG. 2B—Schematic diagram of the caspase-sensitive rtTA. FIG. 2C—Flow chart of using the Caspase Tracker rtTA biosensor system to detect anastasis. FIG. 2D—Time-lapse live cell confocal microscopy of HeLa cell expressing the mammalian CaspaseTracker rtTA biosensor before (i), during (ii-iii) and after (iv-vii) exposure to 1 μM staurosporine in cell culture medium. Merged images of DIC and DsRed signals. FIG. 2E—Time-lapse live cell confocal microscopy of the untreated biosensor-expressing HeLa cells. Cells were incubated with 1 ug/ml doxycycline throughout the experiments of (I)) and (E).

FIG. 3A-3J. Drosophila Caspase Tracker dual biosensor system (reprinted with permission from Tang et al., 9 SCIENTIFIC REPORTS 9015 (2015)). FIG. 3A—Schematic diagram of the Drosophila CaspaseTracker Gal4 biosensor system. FIG. 3B—Schematic diagram of the caspase-sensitive (DQVD) and caspase-insensitive control (DQVA) Gal4. FIG. 3C—Schematic of Drosophila ovary, and flow chart for cell death-induction in 1-day-old flies, followed by 3-days recovery at normal condition. Drosophila ovary drawing provided by Polan Santos; Drosophila image Darren Obbard. FIG. 3D—Representative confocal image of egg chambers from the ovary of female biosensor flies fed with normal fly food for 6 days (untreated). FIG. 3E—Representative confocal image of egg chambers from the ovary of cold shocked female biosensor flies were placed at −7° C. for 1 hour and then switched to normal condition for 1 day (Cold Shock). FIG. 3F—Like panel E except the cold shocked flies were switched to normal condition for 3 days (CS Recovered). FIG. 3G—Representative confocal image of egg chambers from the ovary of starved female biosensor flies fed with 8% sucrose in 1% agar without protein for 3 days to induce apoptosis in egg chambers (Starved). FIG. 3H—Like panel G except the treated flies were switched to normal fly food for 3 days after protein starvation treatment (Re-fed). FIG. 3I—Like panel E except the cold shock of caspase insensitive Caspase Tracker DQVA female biosensor, which served as negative control. FIG. 3J—Confocal image of egg chambers from starved and re-fed female Drosophila. Arrows indicate nuclear GFP expressing in the nurse cells (black), oocyte (white) and follicle cells (yellow) of egg chambers, and in the germarium (green).

FIG. 4. Physiological, pathological and therapeutic implications of anastasis (reprinted with permission from Tang et al., 6 F1000RES 43 (2017).

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Anastasis (Greek for “rising to life”) is a recently discovered cell recovery phenomenon whereby dying cells can reverse late-stage cell death processes that are generally assumed to be intrinsically irreversible. Promoting anastasis could in principle rescue or preserve injured cells that are difficult to replace such as cardiomyocytes in heart failure or neurons in brain injury, thereby facilitating tissue recovery. Conversely, suppressing anastasis in dying cancer cells, undergoing apoptosis due to anti-cancer therapies, may promote cancer cell death and reduce the chances of recurrence. However, these studies have been hampered by the lack of tools for tracking the fate of cells that undergo anastasis in live animals. The challenge is to identify and verify that the cells have reversed cell death process despite their morphologically normal appearance after recovery. To overcome this difficulty, we have generated the Drosophila and mammalian CaspaseTracker biosensor systems that can identify and permanently track the anastatic cells in vitro or in vivo. Here, we present in vivo protocols for the generation and use of this newly developed CaspaseTracker dual biosensor systems to detect and track anastasis in Drosophila melanogaster after transient exposure to cell death stimuli. Unlike conventional biosensors and protocols that label cells actively undergoing apoptotic cell death process, the biosensor permanently labels cells that have recovered after caspase activation, a hallmark of late-stage apoptosis. Therefore, this protocol enables us to continuously track the fate of these cells and their progeny, facilitating future studies of the biological functions, molecular mechanisms, physiological and pathological consequences, and therapeutic implications of anastasis. We also discuss the appropriate controls to distinguish cells that undergo anastasis from those that display non-apoptotic caspase activity in vivo.

As used herein, the term “polynucleotide” or “nucleic acid” refers to a polymeric form of nucleotides of any length, either ribonucleotides and/or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of polynucleotides: a gene or gene fragment, exons, introns, mRNA, IRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

The term “promoter” refers to the DNA region, usually upstream of the coding sequence of a gene or operon, which binds RNA polymerase and directs the enzyme to the correct transcriptional start site.

As used herein, the term “vector” refers to a polynucleotide construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors,” which comprise the attributes of more than one type of vector

A “site-specific recombination event” refers to an event catalyzed by a system generally consisting of three elements: a pair of DNA sequences (the site-specific recombination sequences or sites) and a specific enzyme (the site-specific recombinase). The site-specific recombinase catalyzes a recombination reaction only between two site-specific recombination sequences depending on the orientation of the site-specific recombination sequences. Sequences intervening between two site-specific recombination sites will be inverted in the presence of the site-specific recombinase when the site-specific recombination sequences are oriented in opposite directions relative to one another (i.e., inverted repeats). If the site-specific recombination sequences are oriented in the same direction relative to one another (i.e., direct repeats), then any intervening sequences will be deleted upon interaction with the site-specific recombinase. Thus, if the site-specific recombination sequences are present as direct repeats at both ends of vector backbone sequences integrated into a eukaryotic genome, such integration of said sequences can subsequently be removed by interaction of the site-specific recombination sequences with the corresponding site specific recombinase.

A number of different site specific recombinase systems can be used including, but not limited to, the Cre/lox system of bacteriophage P1, the FLP/FRT system of yeast, the Gin recombinase of phage Mu, the Pin recombinase of E. coli, the PinB, PinD and PinF from Shigella, and the R/RS system of Zygosaccharomyces rouxii. Recombinases generally are integrases, resolvases or flippases. Also dual-specific recombinases can be used in conjunction with direct or indirect repeats of two different site-specific recombination sites corresponding to the dual-specific recombinase (WO99/25840). In certain embodiments, site-specific recombinase systems are the bacteriophage P1 Cre/lox and the yeast FLP/FRT and the Z. rouxii R/RS systems. In these systems a recombinase (Cre, FLP or R, respectively) interact specifically with its respective site-specific recombination sequence (lox, FRT or RS respectively) to invert or excise the intervening sequences. The site-specific recombination sequences for each of these two systems are relatively short (34 bp for lox and 47 bp for FRT).

Accordingly, in one aspect, the present invention provides anastasis biosensor constructs. In one embodiment, a construct comprises the nucleotide sequence of SEQ ID NO:1. In another embodiment, a construct comprises the nucleotide sequence of SEQ ID NO:3. In yet another embodiment, a construct comprises the nucleotide sequence of SEQ ID NO:27. Alternatively, a construct can comprise the nucleotide sequence of SEQ ID NO:29. In a specific embodiment, a construct comprises the nucleotide sequence of SEQ ID NO:31. In another embodiment, a construct comprises the nucleotide sequence of SEQ ID NO:33. A construct can also comprise the nucleotide sequence of SEQ ID NO:35. In a further embodiment, a construct comprises the nucleotide sequence of SEQ ID NO:37. In yet a further embodiment, a construct comprises the nucleotide sequence of SEQ ID NO:39.

Alternatively, a construct of the present invention can comprise the amino acid of SEQ ID NO:2. In another embodiment, a construct comprises the amino acid sequence of SEQ ID NO:4. In yet another embodiment, a construct comprises the amino acid sequence of SEQ ID NO:28. Alternatively, a construct can comprise the amino acid sequence of SEQ ID NO:30. In a specific embodiment, a construct comprises the amino acid sequence of SEQ ID NO:32. In another embodiment, a construct comprises the amino acid sequence of SEQ ID NO:34. A construct can also comprise the amino acid sequence of SEQ ID NO:36. In a further embodiment, a construct comprises the amino acid sequence of SEQ ID NO:38. In yet a further embodiment, a construct comprises the amino acid sequence of SEQ ID NO:40. The constructs can comprise a conservative substitution of up to 1, 2, 3, 4, 5, 6, 7, 8, 9, 10, 11, 12, 13, 14, 15, 16, 17, 18, 19, 20, 25, 30, 35, 40, 45, 50, 55, 60, 65, 70 or more amino acids. In particular embodiments, the constructs comprise a conservative substitution of up to 1-25 amino acids.

Alternatively, a tracking construct of the present invention can comprise Lyn11-NES-ERT2-DEVD-rtTA-3×FLAG-DEVD-ERT2-NES. In another embodiment, the construct comprises the following elements: Lyn11-NES-ERT2-DEVD-rtTA. In yet another embodiment, the construct comprises Lyn11-NES-DEVD-rtTA. The construct can also comprise Lyn11-NES-ERT2-DEVD-rtTA-3×FLAG. In a specific embodiment, a construct comprises Lyn11-NES-DEVD-rtTA-3×FLAG. In yet another embodiment, a construct comprises MCD8-NES-DEVD-rtTA. In a further embodiment, a construct comprises ERT2-DEVD-rtTA-3×FLAG-DEVD-ERT2.

In certain embodiments, the present invention utilizes a split transcription factor/transactivator, for example, split rtTA. The split system would only be activated when both mitochondrial outer membrane permeabilization and caspase-3 activation occurs. For example, the N-terminal end of a transactivator like rtTA can be brought to the mitochondrial intermembrane space using a mitochondrial targeting sequence. In one embodiment, a MTS comprises Mito-CAVP (SEQ ID NO:40 (nucleotide) or 41 (amino acid)). A construct described herein can comprise the C-terminal end of rtTA. When apoptosis occurs, the functional transactivator is formed and the reporter system is activated. Thus, in certain embodiments, a construct of the present invention comprises a first half of a split transactivator and another construct comprises the second half of the transactivator coupled with a MTS.

Examples of the split approach include Split Gal4 (Refined spatial manipulation of neuronal function by combinatorial restriction of transgene expression., Neuron. 2006 52(3) 425-36, Luan H, Peabody N C, Vinson C R, White B H), Split Q (Controlling gene expression with the Q repressible binary expression system in Caenorhabditis elegans., Nat Methods. 2012 9(4) 391-5, Wei X, Potter C J, Luo L, Shen K), Split Cre (Split-CreERT2: temporal control of DNA recombination mediated by split-Cre protein fragment complementation, PLOS One. 2009 Dec. 16; 4(12): e8354, Hirrlinger J, Requardt R P, Winkler U, Wilhelm F, Schulze C, Hirrlinger P G), Split FLPase (Reconstruction of Split-recombinase FLP and Its Recombination Activation in Transgenic Tobacco; Sequential gene targeting to make chimeric tumor models with de novo chromosomal abnormalities., Cancer Res. 2014 74(5) 1588-97, Chambers J S, Tanaka T, Brend T, Ali H, Geisler N J, Khazin L, Cigudosa J C, Dear T N, MacLennan K, Rabbitts T H), and Split Intein (Intersectional Cre driver lines generated using split-intein mediated split-Cre reconstitution., Sci Rep. 2012 2 497, Wang P, Chen T, Sakurai K, Han B X, He Z, Feng G, Wang F.).

In particular embodiments, sensitivity of the biosensor can be increased by increasing the copy number of the caspase cleavage sequence in the linker peptide. In other embodiments, the sensitivity of the biosensor can be adjusted, up or down, by fusing the biosensor with a mutant estrogen ligand-binding domain (ERT2)₆₅ or expressing the biosensor using a tetracycline-responsive promoter66, so that the expression level (sensitivity) of the biosensor will depend on the concentration of tamoxifen or tetracycline, respectively. In further embodiments, the sensitivity of the biosensor can be lowered by using other DEVD-containing linker peptides that are less cleavable then the PARP domain-based linker (Poreba et al., 5 COLD SPRING HARB. PERSPECT. BIOL. A008680 (2013), Takemoto et al., 160 J. CELL BIOL. 235-43 (2003), and Talanian et al., 272 J. BIOL. CHEM. 9677-82 (1997).

Thus, in certain embodiments, a biosensor of the present invention comprises at least one construct described herein and a reporter system. A reporter system can comprise (1) a first nucleic acid encoding flippase operably linked to the upstream activating sequence that binds the transactivator; and (2) a second nucleic acid comprising an FRT-flanked stop codon cassette separating a constitutive promoter and a fluorescent protein open reading frame. The fluorescent protein comprises green fluorescent protein, red fluorescent protein, or yellow fluorescent protein. In other embodiments, the reporter system comprises the G-TRACE reporter system. In further embodiments, the transactivator can comprise a recombinase. In such embodiments, the reporter system can comprise a nucleic acid encoding a reporter gene operably linked to a promoter, wherein the recognition target sequence of the recombinase flanks a stop codon cassette located between the reporter gene and the promoter. In other embodiments, a reporter system comprises (1) a first nucleic acid encoding a site specific recombinase operably linked to the site specific sequence for the transcription factor; and (2) a second nucleic acid comprising a stop codon cassette flanked by site specific recombination sequences, wherein the stop codon cassette and flanking sequences separate a constitutive promoter and a fluorescent protein open reading frame.

The transactivators can comprise a transcription factor such as Gal4 or Q, a recombinase such as Cre, FLP or FLPo, or an intein (including split versions of the foregoing). See International Patent Application No. PCT/US2017/061973, incorporated by reference herein.

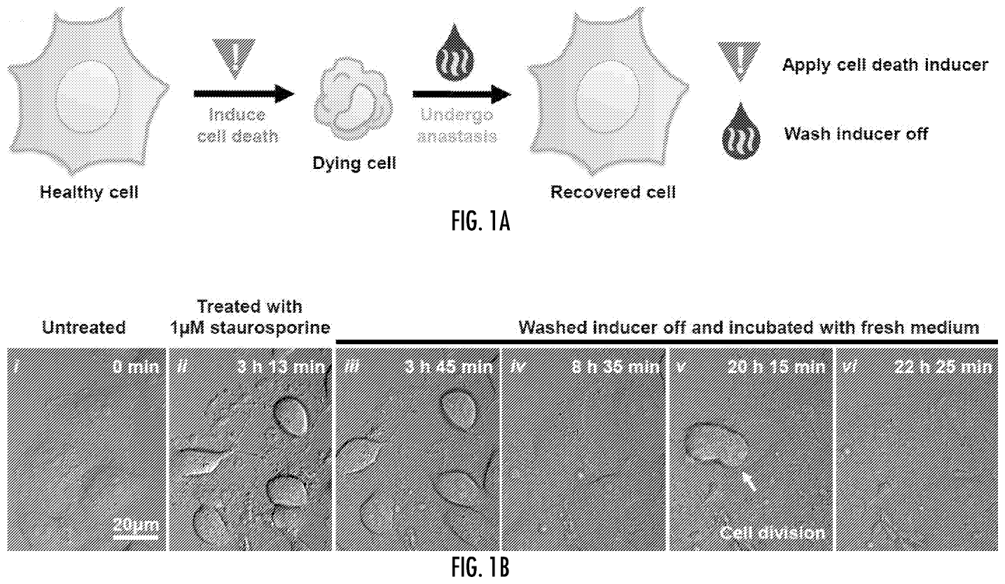

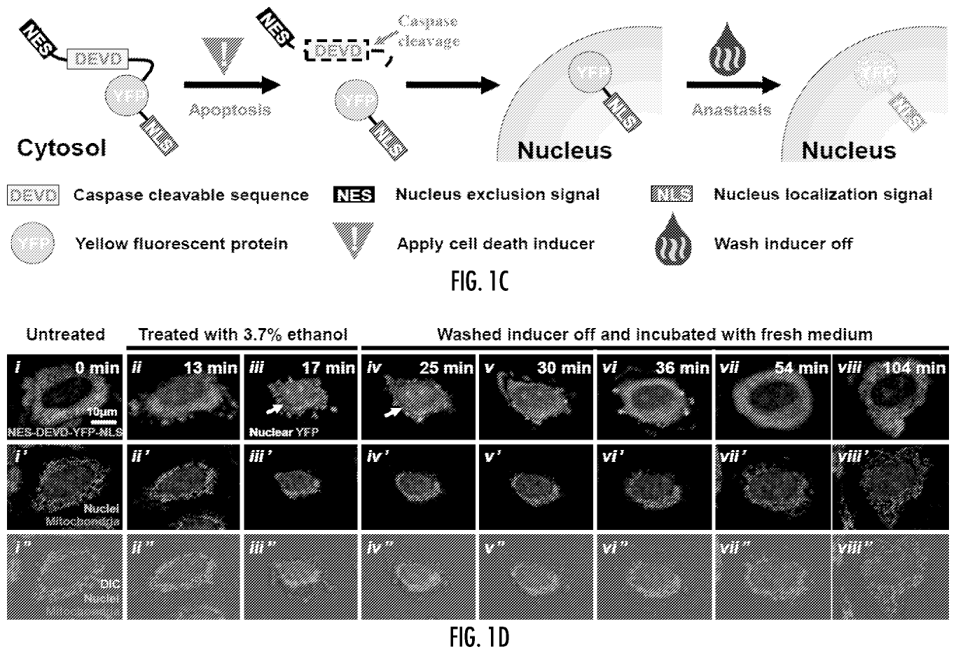

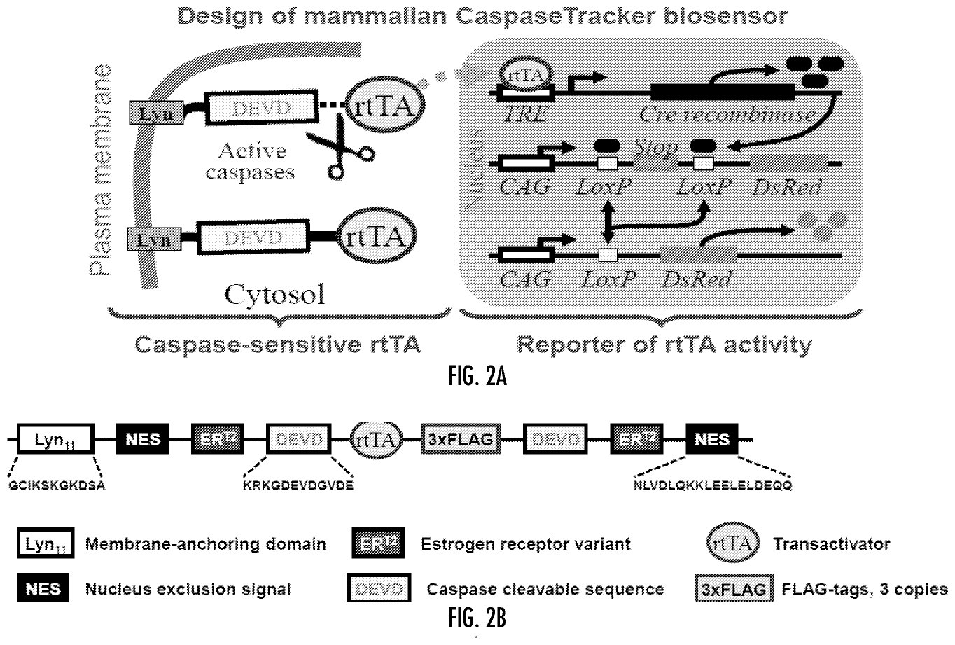

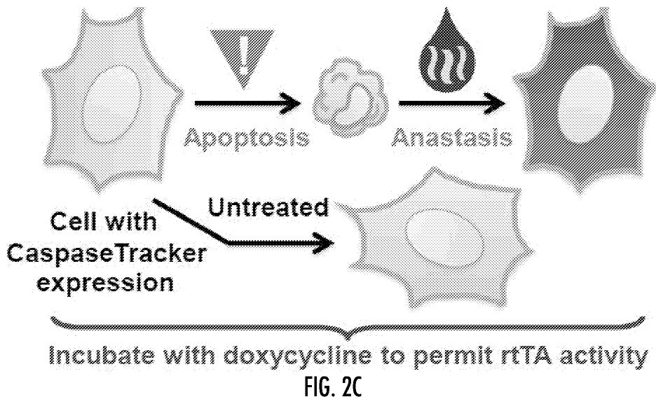

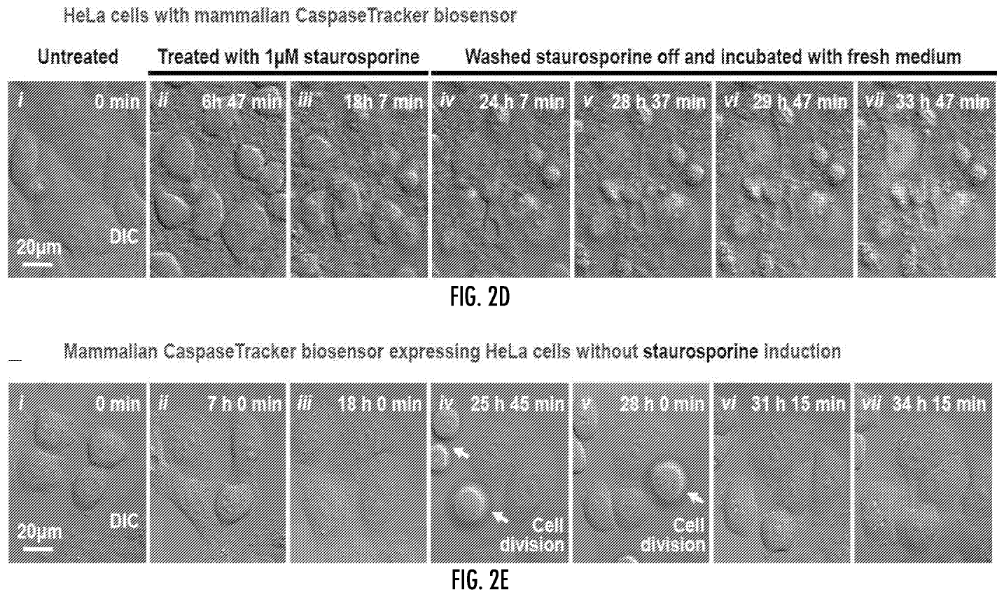

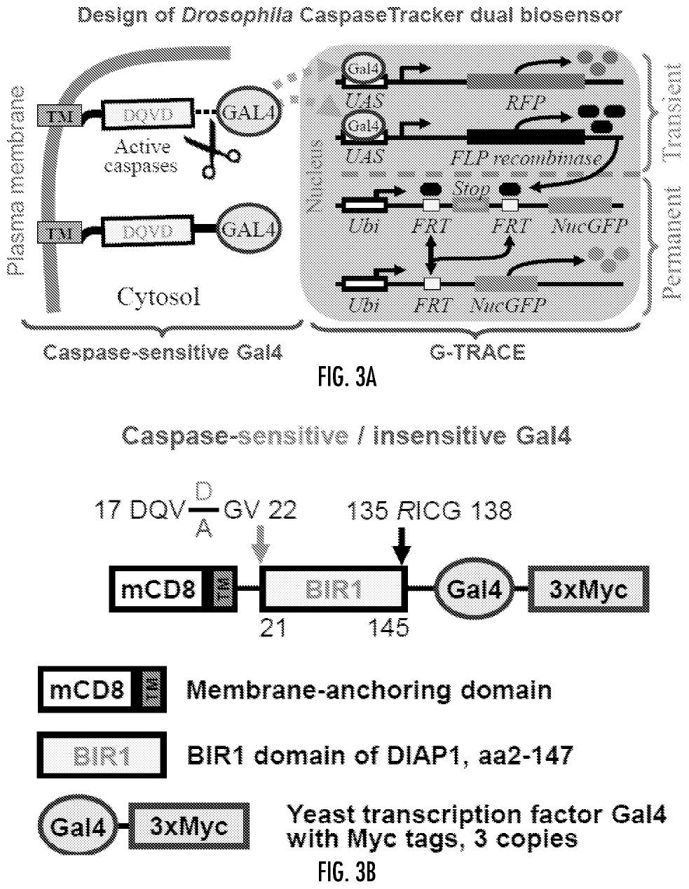

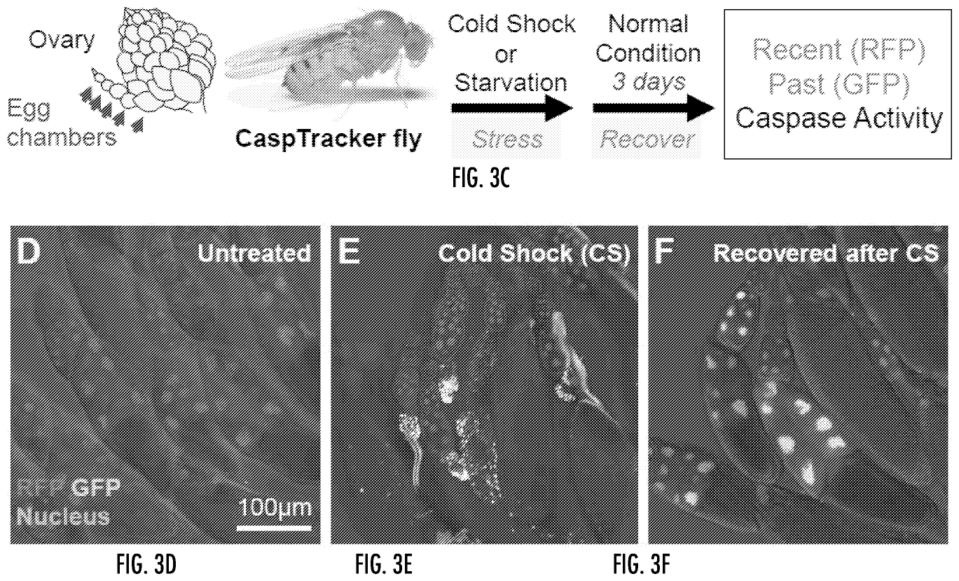

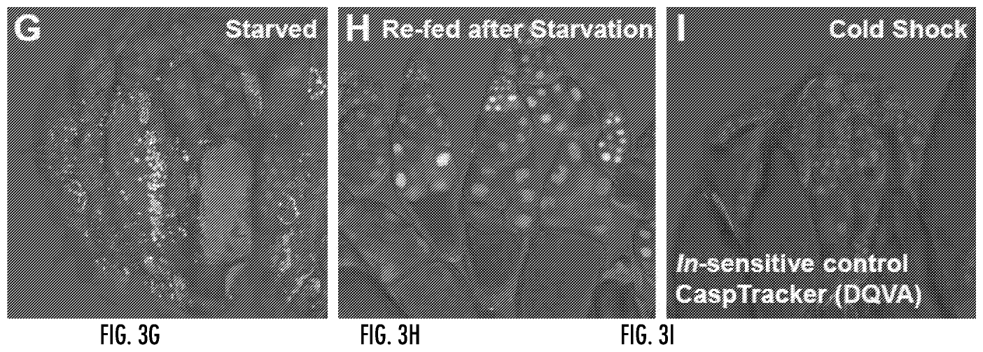

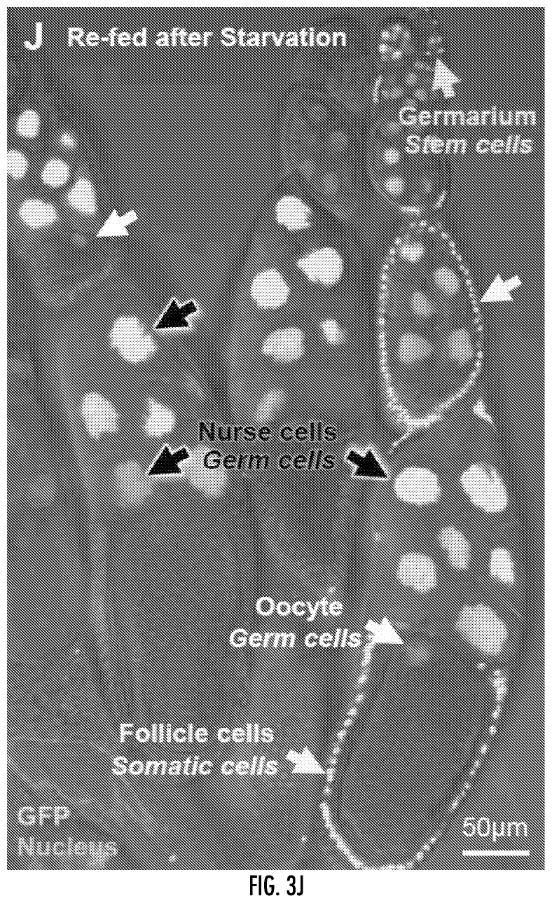

In particular embodiments, the biosensor of the present invention can also comprise a transient reporter in the nucleus. For example, the dual biosensor of the Caspase Tracker (Tang et al., 5 SCI. REP. 9015 (2015) (see FIG. 1 of Tang et al.)) can be used to signal ongoing or transient, as well as permanent reporter expression. Thus, a biosensor can comprise a construct described herein and a reporter system comprising a transient reporter and a permanent reporter. See FIG. 3A. In certain embodiments, a system can comprise a biosensor described herein, a reporter system as well as a control as described in FIG. 3B.

In particular embodiments, rtTA is replaced by another non-mammalian transcription factor or transactivator. In one embodiment, a construct comprises the following elements: Lyn11-NES-ERT2-DEVD-transactivator-3×FLAG-DEVD-ERT2-NES. In another embodiment, the construct comprises the following elements: Lyn11-NES-ERT2-DEVD-transactivator. In yet another embodiment, the construct comprises Lyn11-NES-DEVD-transactivator. The construct can also comprise Lyn11-NES-ERT2-DEVD-transactivator-3×FLAG. In a specific embodiment, a construct comprises Lyn11-NES-DEVD-transactivator-3×FLAG. In yet another embodiment, a construct comprises MCD8-NES-DEVD-transactivator. In a further embodiment, a construct comprises ERT2-DEVD-transactivator-3×FLAG-DEVD-ERT2.

A construct of the present invention can comprise a transactivator and one or more of the following: a transmembrane domain (TD), a nuclear exclusion signal, ERT2, caspase cleavable linker, and purification tag (e.g., FLAG, 3×FLAG, HIS, 6×HIS).

The present invention also provides methods for using the biosensors and reporting systems described herein. Such methods can include methods for studying anastasis and are described in the Examples section below.

In further embodiments, the biosensors of the present invention can be used for drug screening. In certain embodiments, the biosensors can be expressed in organoids from a patient. Drugs that kill cancer cells without anastasis can be screened. If the biosensor indicates that anastasis is occurring or likely to occur, then other drugs can be used or an anastasis inhibitor could also be used to prevent cancer recurrence during and after drug treatment. In alternative embodiments, the screening can take place using patient-derived xenograft mice. See, e.g., Pauli et al., 7(5) CANCER DISCOV. 462-77 (2017).

Without further elaboration, it is believed that one skilled in the art, using the preceding description, can utilize the present invention to the fullest extent. The following examples are illustrative only, and not limiting of the remainder of the disclosure in any way whatsoever.

### Examples

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Anastasis is technically challenging to be detected in vivo because the cells that have reversed cell death process can be morphologically indistinguishable from the normal healthy cells. Here we demonstrate protocols for detecting and tracking cells that undergo anastasis in live animals using our newly developed in vivo CaspaseTracker dual biosensor systems.

### Protocol

### 1) Preparation of CaspaseTracker Biosensor Flies

1.1. Anesthetize flies with CO₂, and use a paintbrush to transfer 7 to 10 caspase-sensitive Gal4 (DQVD)¹⁶ virgin females and 7 to 10 young G-Trace (Evans et al., 6 NAT. METHODS 603-05 (2009)) Gal4 reporter male flies (or vice versa) in the same vial with fly food and fresh yeast paste.

NOTE: Cross of Caspase-sensitive (DQVD) Gal4 and G-Trace flies will produce CaspaseTracker progeny flies. Cross of Caspase-insensitive (DQVA) Gal4 and G-Trace flies will provide negative control flies (see discussion). Fresh yeast paste serves as protein source to enhance egg production, so that increases number of progeny.

1.2. Incubate the files at 18 degrees Celsius (° C.) for 3 to 7 days, and then transfer the flies to new vial to set up a new cross at 18° C. Continue to incubate only the original vial at 18° C. until progeny flies eclose.

NOTE: Transfer the parent flies to new vials to avoid overcrowding of progeny at the original vial. Parent files can produce progeny with fresh food and yeast paste at the first 2 to 3 switches, and then the productivity will significantly decrease with time. Raising files 18° C. can reduce non-specific signal of CaspaseTracker biosensor (see Discussion).

1.3. Select progeny flies with correct phenotypes for following experiments.

NOTE: The transgenes of both caspase-sensitive Gal4 and G-Trace are located at the second chromosome, balanced with CyO balancer. Select the non-curly wing progeny (without CyO), which has both transgenes of caspase-sensitive Gal4 and G-Trace.

### 2) Application of Transient Apoptotic Induction to CaspaseTracker Biosensor Flies

2.1. Transfer 10 to 20 newly enclosed female flies to new vial with fresh fly food and fresh yeast paste for 1 day at 18° ° C. to allow egg chamber production by oogenesis.

NOTE: Keeping the female with male flies might enhance egg chamber production.

2.2. To induce egg chambers to undergo apoptosis by cold shock, transfer the female flies to new vial, which is then placed at −7° C. for 1 hour.

2.3. To induce egg chambers to undergo apoptosis by protein starvation, transfer the female flies to a new vial with 8% sucrose and 1% agar food at 18° C. for 3 days.

NOTE: Protein starvation (non-protein food) can trigger egg chambers to undergo apoptosis (Jenkins et al., 23 TRENDS CELL. BIOL. 567-74 (2013); Drummond-Barbosa, D. & Spradling, A. C., 231 DEV. BIOL. 265-78 (2001); Pritchett et al., 14 APOPTOSIS 969-79 (2009)). Switch flies to new vial with 8% sucrose and 1% agar food every day to keep optimal condition of the sucrose fly food.

2.3. Transfer the stressed flies back to new vial with fresh fly food and fresh yeast paste for 3 day at 18° C. to allow them to recover. Dissect the starved and the starved-recovered flies to obtain egg chambers at ovaries as described (Wong, L. C. & Schedl, P., 51 J. VIS. EXP. (2006)).

NOTE: To dissect Drosophila to obtain ovaries, anesthetize flies with CO₂, and use 2 pairs of forceps to remove fly head, and the use the forceps to pull the base of the abdomen to remove the ovaries of the flies.

### 3) Fixation and Staining of Dissected Egg Chambers for Imaging

3.1. Transfer the dissected egg chambers together with around 0.5 mL phosphate buffered saline (PBS) to 1 mL centrifuge tubes. Allow the eggs to settle down.

NOTE: Coat the plastic pipette tips with 1% bovine serum albumin (BSA) dissolved in water or PBS to prevent the egg chambers to stick at the tips. Perform the following procedures in dark to avoid photobleaching of red fluorescent protein (RFP, also known as DsRed) and green fluorescent protein (GFP) in the egg chambers.

3.2. Remove the PBS by pipetting, and then apply 0.5 mL 4% paraformaldehyde in PBS to fix the egg chambers at room temperature in dark for 20 to 30 minutes. Allow the eggs to settle down.

NOTE: Apply gentle rotation in the following incubation steps.

3.3. Remove the paraformaldehyde by pipetting, and then washed the egg chamber with 0.5 mL PBST (PBS+0.1% Triton X-100) for 3 times. Allow the eggs to settle down. NOTE: Prolonged fixation could reduce the RFP and GFP signals. PBST can avoid egg chambers to stick to the non-BSA coated plastic surface.

3.4. Incubate the egg chambers with PBST overnight at 4° C. with gentle rotation to permeabilize the egg chambers.

3.5. Remove the PBST by pipetting, and then apply 0.5 mL of 10 μg/mL of blue nuclear Hoechst dye in PBST to egg chambers for 1 hour at room temperature to stain for nucleus. Allow the eggs to settle down.

NOTE: Avoid prolonged incubation with nuclear dye as this will increase non-specific signal.

3.6. Remove the nuclear dye by pipetting, and then apply 0.5 mL PBST to wash the egg chambers in the 1 mL centrifuge tubes for 3 times, with 10 minutes incubation with gentle rotation between each washing step. Allow the eggs to settle down.

3.7. Remove all PBST with fine pipette, and then then apply 200 μL anti-bleaching mounting agent (see materials) to incubate the egg chambers for 3 hours or overnight until the egg chambers sink to the bottom of the tube.

3.8. Mount the stained egg chambers by transferring them with 200 μL anti-bleaching mounting agent on glass slide for imaging by pipetting, cover the egg chambers with 20×20 mm glass cover slip, and seal the cover slip on glass slide by putting nail polish at the edge of the cover slip.

3.9. Image the egg chambers using fluorescence or confocal microscope, using a 20×, NA 0.8 Plan-Apochromat objective, with excitation light wavelength 405 nm for nuclear staining (detect emission ˜461 nm), 561 nm for RFP (ongoing or recent caspase activity) signal (detect emission ˜570 nm), and 488 nm for GFP (past caspase activity) signal (detect emission ˜518 nm).

### Results

While time-lapse live cell microscopy is a reliable method to tract anastasis in cultured cells (Tang et al., 96 J. VIS. EXP. 51964 (2015)), it is challenging to identify which cells have undergone anastasis in animals, because the recovered cells appear morphologically indistinguishable from the normal health cells that have not attempted cell death. For example, human cervical cancer HeLa cells display morphological hallmarks of apoptosis (Taylor et al. (2008); Jacobson et al. (1997); Kerr et al. (1972), such as cell shrinkage, nuclear condensation, and plasma membrane blebbing in response to cell death stimulus 1 μM staurosporine (FIG. 1A, FIG. 1Bi-ii). After removal of the cell death stimulus and incubated in fresh medium, the dying cells reverse the cell death process by anastasis (Tang et al. (2017); Tang et al. (2015); Tang et al. (2012); Tang et al. (2009), as indicated by morphological recovery (FIG. 1Biii-iv), followed by proliferation (FIG. 1Bv-vi). Our previous studies have also used the “real-time” caspase biosensors, such as ApoAlert (NES-DEVD-YFP-NLS) to demonstrate reversal of apoptosis after caspase activation (Tang et al. (2015); Tang et al. (2012). This biosensor localized at cytosol in healthy cells (FIGS. 1C, 1Di). Upon caspase activation triggered by cell death stimulus 3.7% ethanol, this YFP-based biosensor is cleaved by caspases, and then translocate to nucleus, so that it can label the cells displace on-going caspase activity by nuclear YFP (FIGS. 1C, 1Dii-iii). The apoptotic cell also displays morphological hallmarks of apoptosis during ethanol-induction (Taylor et al. (2008); Kerr et al. (1972)), such as fragmentation of tubular mitochondria, nuclear condensation, cell shrinkage, and plasma membrane blebbing (FIG. 1Dii-iii). Interestingly, after removal of the cell death stimulus, the same cell can recover, and regain normal morphology (FIG. 1Biv-vii). Noticeably, the nuclear signal of the ApoAlert biosensor is removed within 1 hours in the recovered cell (FIG. 1Div-vii), possibly by the same mechanisms of the anastatic cells to remove the damaged cell components (Tang et al. (2012), such as cleaved caspase-3 and PARP generated during apoptosis. Therefore, new strategy is required for tracking anastasis in long term, especially in vivo.

To detect, label and track anastatic cells in live animals, we develop the mammalian CaspaseTracker biosensor system. This biosensor is composed of caspase-sensitive rtTA, and Cre-LoxP-mediated rtTA activity-dependent reporter system (FIG. 3A). In the healthy cells without caspase activity, the transactivator rtTA (reverse tetracycline-controlled transactivator) (Gossen et al., 268 SCIENCE 1766-69 (1995)) is tethered to plasma membrane anchor (Lyn11) (Inoue et al., 2 NAT. METHODS 415-18 (2005); Yamanashi et al., 7 MOL. CELL. BIOL. 237-43 (1987)), nucleus exclusion signal (NES) of Map Kinase (MAPKK)(Fukuda et al., 271 J. BIOL. CHEM. 20024-28 (1996)), and estrogen receptor variant (ERT2) (Feil et al., 237 BIOHCEM. BIOPHYS. RES. COMMUN. 752-57 (1997)) through caspase-cleavable (DEVD) (Lazebnik et al., 371 NATURE 346-67 (1994)) linkers derived from PARP (FIGS. 3A, 3B). As tethered rtTA cannot translocate from cytosol to nucleus, the rtTA reporter remains inactive. Upon caspase activation in response to cell death stimulus, activated caspases cleave the DEVD linkers to release rtTA, which then translocates to the nucleus to activate the rtTA reporter (FIG. 3A). The nuclear rtTA binds to the tet response element (TRE) to triggers transient expression of Cre recombinase, which leads to an irreversible recombination event that removes the stop cassette between the CAG promoter and the coding sequences for red fluorescent protein (DsRed). This results in permanent expression of DsRed, which serves as the permanent fluorescent marker of those cells that can remain alive after they have experienced caspase activity, as well as their progeny (FIG. 3C). Doxycycline, which is essential to permit rtTA activity, is applied at the time when the biosensor need to be turn on.

To test the mammalian Caspase Tracker biosensor, we introduce the biosensor to the HeLa cells by transient transfection, expose the cells with transient cell death stimulus, and monitor the recovery of the cells by time-lapse live cell confocal microscopy as we described (Tang et al., 96 J. VIS. EXP. 51964 (2015)). Doxycycline (1 μg/ml) is applied to the cell culture medium to turn on the biosensor throughout the experiment. In response to the cell death stimuli 1 μM staurosporine, the induced cells display hallmark of apoptosis including cell shrinkage and plasma membrane as expected (FIG. 3Di-iii). After wash and incubate the cells with fresh culture medium, anastasis occurs in the dying cells, as induced by their morphological recovery (FIG. 3Div-viii). Importantly, the recovered cells express DsRed during and after anastasis (FIG. 3Div-viii), but not the non-recovered cells (FIG. 3Div-viii), nor the cells without exposed to cell death stimuli (FIG. 3E). This indicates that the mammalian CaspaseTracker is a unique tool to identify and permanently label the cells that recovered from caspase-activation, enabling the long-term track to study the fate of anastatic cells.

To detect and track anastasis in live animals, the CaspaseTracker biosensor transgenic animals are first created and tested in Drosophila melanogaster (Tang et al., 5 SCI. REP. 9015 (2015)). Modified from the mammalian CaspaseTracker biosensor, the Drosophila dual biosensor is composed of a caspase-sensitive Gal4 (Tang et al. (2015), and the Gal4 reporter G-Trace (Evans et al. (2009)) (FIG. 3A). In the cells without caspase activity, the yeast transcription factor Gal4 is tethered to a plasma membrane anchor (mCD8) domain through a caspase-cleavable linker (DQVD) derived from DIAP1 (FIG. 3B), with a D135R mutation to abolish the drICE caspase inhibitory function in the BIRI domain¹⁶. As tethered Gal4 cannot translocate to nucleus, the Gal4 reporter G-Trace remains inactive in the cells without caspase activity. Upon caspase activation, activated caspases cleave the DQVD linker to release Gal4, which then translocates to the nucleus to activate the G-Trace reporter (FIG. 3A). Gal4 binds to the specific upstream activating sequences (UAS) to triggers transient expression of RFP, which serves as the reporter of recent or current caspase activity until the Gal4 (caspase) activity stop and then RFP protein is degraded. Gal4 also triggers the expression of FLP recombinase, which leads to a recombination event that removes the stop cassette between the ubiquitin (Ubi) promoter and the coding sequences for nuclear-targeted GFP (nucGFP). This results in permanent expression of nucGFP, which serves as the permanent marker of those cells that have experienced caspase activity and remain alive. Transgenic CaspaseTracker Drosophila is generated with this dual biosensor system.

To test the Drosophila CaspaseTracker biosensor for detecting apoptosis and anastasis in vivo, the CaspaseTracker female flies are subjected to physiological stress (FIG. 3C) such as cold shock (Tang et al., 5 SCI. REP. 9015 (2015)), which can efficiently trigger cell death including apoptosis in various tissues such as egg chambers of fruit flies (Yi et al., 12 APOPTOSIS 1183-93 (2007)). As expected, CaspaseTracker is not activated at the healthy egg chambers of well-fed flies as apoptosis is not occurred (FIG. 3D), but is activated in the stressed flies in 1 day after cold shock induction (FIG. 3E). Egg chambers exhibiting apoptotic morphologies, including cell shrinkage and nuclear condensation, display RFP and GFP biosensor activity, indicating recent or on-going (RFP) and past (GFP) caspase activity (FIG. 3E). However, at 3 days after the flies are kept in normal condition, the GFP, but not RFP, express in the egg chambers (FIG. 3F), inducing that the egg chambers experienced the past caspase activity, and survive.

To further test reversibility of cell death process in egg chambers (Tang et al. (2015)), CaspaseTracker female flies are fed with 8% sucrose in 1% agar for 3 days, as previous studies demonstrate that protein starvation can trigger caspase-mediated apoptosis in tissues with somatic and germ cells including egg chambers (Drummond-Barbosa, D. & Spradling, A. C., 231 DEV. BIOL. 265-78 (2001)). As expected, CaspaseTracker is activated in egg chambers after 3 days of protein starvation (FIG. 3G). The dying egg chambers exhibiting apoptotic morphologies display RFP and GFP biosensor activity, indicating recent or on-going (RFP) and past (GFP) caspase activity (FIG. 3G). To demonstrate that CaspaseTracker can track the recovered cells that previously experienced caspase activation after a death stimulus, the starved flies are then transferred to normal protein-containing fly food. As expected, the recovered egg chambers of these re-fed flies lack the RFP transient caspase reporter, indicating no recent or ongoing caspase activity (FIG. 3H). However, these re-egg chambers display the GFP caspase reporter (FIG. 3H), indicating that the cells in these egg chambers have reversed apoptosis at a point after caspase activation. The CaspaseTracker biosensor activity is triggered by caspase activity, because replacing the caspase cleavable DQVD with caspase non-cleavable DQVA peptide at the biosensor abolish its activity (FIG. 3I).

After the Caspase Tracker Drosophila recovered from protein starvation, we found that multiple cell types of egg chambers, such as somatic (follicle) cells and germline cells (nurse cells and oocytes), display only GFP, but not RFP (FIG. 3J) (Tang et al., 5 SCI. REP. 9015 (2015)), indicating that these cells can undergo anastasis after caspase activation. Importantly, the starved and re-fed female flies lay fertile eggs that can produce GFP-expressing progeny flies (Tang et al. (2015)), suggesting that potentially many cells can reverse cell death process after caspase activation and regain apparently normal function. Future studies are needed to determine if progeny flies that survive as a consequence of anastasis exhibit permanent sequelae.

### Discussion

The CaspaseTracker dual biosensor system is a novel and unique tool that allows detection of recent or ongoing caspase activity, and tracking of cells that have reversed apoptosis and survive after experiencing caspase activity in vivo. While caspase activity is traditionally known as the hallmark of apoptosis, recent studies reveal that non-apoptotic caspase activity plays potential roles in diverse normal cell functions, such as regulation of neuronal activity (Li et al., 141 CELL. 859-71 (2010); Jonas et al., 101 PROC. NATL. ACAD. SCI. U.S.A. 13590-95 (2004)), learning and memory (Neukomm, L. J. & Freeman, M. R., 24 TRENDS CELL. BIOL. 515-23 (2014); Yu, F. & Schuldiner, O., 27 CURR. OPIN. NEUROBIOL. 192-98 (2014); Maor-Nof, M. & Yaron, A., 23 CURR. OPIN. NEUROBIOL. 990-96 (2013); Hyman, B. T. & Yuan, J., 13 NAT. REV. NEUROSCI. 395-406 (2012)), suppression of necroptotic cell death (Kaiser et al., 471 NATURE 368-72 (2011); Oberst et al., 471 NATURE 363-67 (2011)), spermatid individualization (Kaplan et al., 19 DEV. CELL 160-73 (2010); Arama et al., 4 DEV. CELL 687-97 (2003)), and microRNA processing (Weaver et al., 3 ELIFE (2014)). In addition to apoptosis and anastasis, the CaspaseTracker biosensor system can detect non-apoptotic caspase activity, which presents in brain and optic lobes, cardia, guts, Malpighian tubules, trachea, and other tissues (Tang et al., 117 J. VIS. EXP. (2016); Tang et al., 5 SCI. REP. 9015 (2015)). To study anastasis in live animals, it is critical to choose the tissues with cells that exhibit no caspase biosensor activity under normal physiological conditions, but can be induced to undergo caspase activation by transient cell death induction. Egg chambers are ideal, because they often do not have caspase activity from germarium to stage 10 during oogenesis (Jenkins et al., 23 TRENDS CELL. BIOL. 567-74 (2013); Pritchett et al., 14 APOPTOSIS 969-79 (2009); Baum et al., 14 CELL DEATH DIFFER. 1508-17 (2007)).

Exposing female Drosophila to transient environmental stresses, such as protein starvation and cold shock, can efficiently trigger apoptosis in egg chambers (Pritchett et al. (2009); Chaturvedi et al., 99 J. NATL. CANCER INST. 1634-43 (2007); Yi et al. (2007)). Critical steps within the protocol include avoiding prolonged apoptotic induction to flies. The optimized conditions of protein starvation (8% sucrose in 1% agar for 3-days) and cold shock (1 hour at −7° C.) to female flies can trigger caspase-mediated apoptosis in egg chambers, and allow them to recover after the stressed flies are returned to normal condition (Tang et al., 5 SCI. REP. 9015 (2015)). Prolonged cell death stimulus can trigger more egg chambers to undergo apoptosis, but the recovery rate is also reduced, possibly because the dying egg chambers experience massive damage beyond repair.

An addition critical step in this protocol is to reduce the CaspaseTracker background signal in egg chambers by crossing, raising and maintaining the CaspaseTracker flies at low temperature such as 18° C. While the majority of egg chambers from optimally reared flies do not display caspase activity in the germarium through stage 10 during oogenesis (Pritchett et al. (2009)), an around 1% of egg chambers could exhibit caspase biosensor activity without cell death induction. This may reflect the normal attrition rate due to innate errors or may be triggered un-intentionally during oogenesis by standard laboratory conditions. As Gal4 displays less activity in flies at low temperature (Duffy, J. B., 34 GENESIS 1-15 (2002)), raising flies at 18° C., rather than at room temperature, can reduce the endogenous signal that activates the CaspaseTracker system. Alternatively, switching files to high temperature, such as 29° C., can increase sensitive of CaspaseTracker system, due to increase in Gal4 activity (Duffy et al. (2002)), and potentially other endogenous temperature-dependent enzymatic activities.

It is important to distinguish the CaspaseTracker-labeled cells that undergo apoptosis and anastasis from cells that exhibit non-apoptotic caspase activity. Apoptotic cells express RFP, and often GFP in prolonged apoptotic induction, as the cells has ongoing caspase activity that cleave-activated Gal4, which activate the transient (Gal4 activity-dependent RFP) and permanent (Gal4 triggered FLPase-FRT mediated GFP) labeling reporters at the G-Trace system. Apoptosis of these cells can be confirmed by morphological hallmark such us nuclear condensation stained with nuclear dye (Pritchett et al. (2009); Taylor et al. (2008)), and also biochemical hallmark for cleaved caspases by immunostaining (Fan, Y. & Bergmann, A., 17 CELL DEATH DIFFER. 534-39 (2010)). For the cells that reversed apoptosis, they display permanent GFP expression due to the FLPase-mediated recombination event of G-Trace system. These cells don't have RFP expression as the cells don't have on-going caspase activity, nor other hallmarks of apoptosis (Tang et al., 5 SCI. REP. 9015 (2015)). These cells also display normal nuclear morphology. The cells that have on-going non-apoptotic caspase activity often display both RFP and GFP expression, with normal nuclear morphology (Tang et al. (2015)).

At the same time, it could be difficult to distinguish the cells that experienced anastasis, and the past non-apoptotic caspase activity, because both of the cells only display GFP, and with normal nuclear morphology. Therefore, careful control experiments are needed to be included (Tang et al. (2015)). For examples, to study anastasis in egg chambers, it is essential to examine the GFP expression at both of the stressed-recovered flies and the non-stressed flies (negative control). The recovered flies should more GFP-expressing cells than the non-stressed flies, if anastasis occurs in the recovered cells and tissues. Besides, it is also important to distinguish the signal of CaspaseTracker from nonspecific signal of auto-fluorescence such as from cuticle and fat bodies. We generated the negative control biosensor flies, with only different in DQVD to DQVA mutation to abolish the caspase sensitivity of the control biosensor (Tang et al. (2015)). The signal presents at the caspase sensitive (DQVD) but not the negative control (DQVA) biosensor flies is the real signals triggered by caspase activity, rather than auto-fluorescence.

Our current Drosophila dual Caspase Tracker biosensor can identify the cells with “recent” caspase activity by the RFP, and the cells with “past” caspase activity by GFP (Tang et al. (2015)). At the same time, the RFP is not the “real-time” caspase activity indicator, because it takes a few hours of reaction time for Gal4 to drive the expression of RFP in response to caspase activity. To add the “real time” function, our Drosophila CaspaseTracker biosensor can be combined with the recently developed iCasper biosensor (To et al., 112 PROC. NATL. ACAD. SCI. U.S.A. 3338-43 (2015)), a “real-time” and “dark to bright” in vivo biosensor that only show far red signal when it is cleaved by caspases.

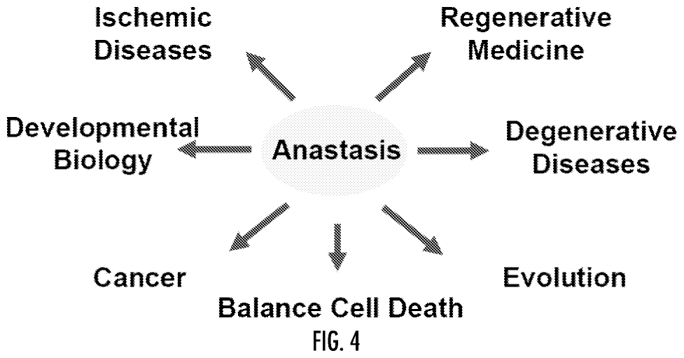

The in vivo CaspaseTracker biosensor will facilitate pursuit of the yet unknown functions, mechanisms and therapeutic implications of anastasis (FIG. 4). To reveal the molecular signature of anastasis, we performed the time-course whole-genome gene expression microarray study to analyze the mouse primary liver cell during reversal of ethanol-induced apoptosis, and interestingly found striking changes in transcription of genes involved in multiple pathways including pro-survival, DNA damage and stress response, angiogenesis, cell migration and transformation (Tang et al. (2017); Tang et al. (2012)). Our finding is supported by our RT-PCR validation during the recovery of the human liver cancer HepG2 cells (Tang et al. (2017)), and also the following independent study in Hela cells (Sun et al., J. CELL. BIOL. jcb.201706134 (2017)). To study the physiological, pathological and therapeutic potentials of anastasis, it is important to identify the anastatic cells and track their fate in small animals. Our Drosophila and mammalian CaspaseTracker biosensors would be the useful tools to test the potential contributions of anastasis in tissue recovery, stress-induced mutagenesis, tumor evolution, cancer recurrence and metastasis. The finding could offer potential to identify revolutionary new therapeutic approaches for intractable diseases by mediating cell death and survival through controlling anastasis.

We claim:

1. A polynucleotide comprising SEQ ID NO:1.

2. A polynucleotide comprising SEQ ID NO:3.

3. A polynucleotide comprising SEQ ID NO:27.

4. A polynucleotide comprising SEQ ID NO:29.

5. A polynucleotide comprising SEQ ID NO:31.

6. A polynucleotide comprising SEQ ID NO:33.

7. A polynucleotide comprising SEQ ID NO:35.

8. A polynucleotide comprising SEQ ID NO:37.

9. A polynucleotide comprising SEQ ID NO:39.

10. A polypeptide comprising SEQ ID NO:2.

11. A polypeptide comprising SEQ ID NO:4.

12. A polypeptide comprising SEQ ID NO:28.

13. A polypeptide comprising SEQ ID NO:30.

14. A polypeptide comprising SEQ ID NO:32.

15. A polypeptide comprising SEQ ID NO:34.

16. A polypeptide comprising SEQ ID NO:36.

17. A polypeptide comprising SEQ ID NO:38.

18. A polypeptide comprising SEQ ID NO:40.

19. A biosensor comprising a polypeptide of any of claims 10-18 and a reporter system.


SEQUENCE LISTING (47 sequences)

SEQ ID NO: 1 - DNA, 2937 bp, synthetic construct
  misc_feature 1..2937: note = Lyn11-NES-ERT2-DEVD-rtTA-3xFLAG-DEVD-ERT2-NES
  source 1..2937: mol_type = other DNA
ggatgtataaaatcaaaagggaaagacagcgcgggagcagatagtgctggtagtgctggtaacctggtggacctccaaaagaagctggaggagctggagctggacgagcagcaggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaagaggaagggcgacgaggtggacggcgtggacgagtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggggactacaaagaccatgacggtgattataaagatcatgacatcgattacaaggatgacgatgacgcaaaacgcaaaggtgatgaagtagatggagtagacgaagcaggggatatgcgcgcagcaaacctctggccttcacccctgatgataaagaggtcaaagaaaaactctctcgctctgtcactcaccgcagatcaaatggtgtccgcactgctggacgccgagcctccaatattgtacagtgaatacgatcccactcggcctttttccgaggcctccatgatggggctcctcacaaatctcgccgatcgagaacttgtgcatatgataaattgggccaaacgagtccctggtttcgttgatcttactctgcacgaccaagtacatctgctggagtgcgcttggctggaaattctcatgatcgggctggtatggaggagcatggaacatcccgtcaaactgctcttcgcccccaacctcttgctcgaccgaaatcaggggaagtgcgtggaagggatggtagaaatatttgatatgctcctggccacaagcagcagattcagaatgatgaacctccaaggcgaagaattcgtttgtttgaagagcatcatcctgctcaacagcggcgtctataccttcctctcctccacgttgaaaagccttgaggaaaaagatcacatccatagggtacttgataaaattactgataccctcatacaccttatggcaaaagcgggactcacacttcaacaacaacatcaaagactcgcacaattgttgttgattttgagccatataaggcatatgtcaaataagggaatggaacacctctactctatgaaatgtaagaatgtcgtcccactgtacgatcttctccttgaagctgcagatgctcatcggctccacgcacctacctctcggggcggcgctagcgttgaagagactgatcagtcacatctcgcgacagctggaagcacaagcagccacagcctccaaaaatactatattacaggcgaggccgaaggcttccccgctacggccgtggataatcttgtagatcttcagaaaaaactcgaagaacttgagcttgatgaacaacaa

SEQ ID NO: 2 - protein, 979 aa, synthetic construct
  REGION 1..979: note = Lyn11-NES-ERT2-DEVD-rtTA-3xFLAG-DEVD-ERT2-NES
GCIKSKGKDSAGADSAGSAGNLVDLQKKLEELELDEQQAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVDKRKGDEVDGVDESRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPGDYKDHDGDYKDHDIDYKDDDDAKRKGDEVDGVDEAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVDNLVDLQKKLEELELDEQQ

SEQ ID NO: 3 - DNA, 2934 bp, synthetic construct
  misc_feature 1..2934: note = Lyn11-Nes-ERT2-DEVD-rtTA-3xFLAG-DEVD-ERT2-Nes minus the gca codon at end of 3x flag
  source 1..2934: mol_type = other DNA
ggatgtataaaatcaaaagggaaagacagcgcgggagcagatagtgctggtagtgctggtaacctggtggacctccaaaagaagctggaggagctggagctggacgagcagcaggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaagaggaagggcgacgaggtggacggcgtggacgagtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggggactacaaagaccatgacggtgattataaagatcatgacatcgattacaaggatgacgatgacaaacgcaaaggtgatgaagtagatggagtagacgaagcaggggatatgcgcgcagcaaacctctggccttcacccctgatgataaagaggtcaaagaaaaactctctcgctctgtcactcaccgcagatcaaatggtgtccgcactgctggacgccgagcctccaatattgtacagtgaatacgatcccactcggcctttttccgaggcctccatgatggggctcctcacaaatctcgccgatcgagaacttgtgcatatgataaattgggccaaacgagtccctggtttcgttgatcttactctgcacgaccaagtacatctgctggagtgcgcttggctggaaattctcatgatcgggctggtatggaggagcatggaacatcccgtcaaactgctcttcgcccccaacctcttgctcgaccgaaatcaggggaagtgcgtggaagggatggtagaaatatttgatatgctcctggccacaagcagcagattcagaatgatgaacctccaaggcgaagaattcgtttgtttgaagagcatcatcctgctcaacagcggcgtctataccttcctctcctccacgttgaaaagccttgaggaaaaagatcacatccatagggtacttgataaaattactgataccctcatacaccttatggcaaaagcgggactcacacttcaacaacaacatcaaagactcgcacaattgttgttgattttgagccatataaggcatatgtcaaataagggaatggaacacctctactctatgaaatgtaagaatgtcgtcccactgtacgatcttctccttgaagctgcagatgctcatcggctccacgcacctacctctcggggcggcgctagcgttgaagagactgatcagtcacatctcgcgacagctggaagcacaagcagccacagcctccaaaaatactatattacaggcgaggccgaaggcttccccgctacggccgtggataatcttgtagatcttcagaaaaaactcgaagaacttgagcttgatgaacaacaa

SEQ ID NO: 4 - protein, 978 aa, synthetic construct
  REGION 1..978: note = Lyn11-Nes-ERT2-DEVD-rtTA-3xFLAG-DEVD-ERT2-Nes minues the gca codon at end of 3x flag
GCIKSKGKDSAGADSAGSAGNLVDLQKKLEELELDEQQAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVDKRKGDEVDGVDESRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPGDYKDHDGDYKDHDIDYKDDDDKRKGDEVDGVDEAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVDNLVDLQKKLEELELDEQQ

SEQ ID NO: 5 - DNA, 33 bp, synthetic construct
  misc_feature 1..33: note = Lyn11
  source 1..33: mol_type = other DNA
ggatgtataaaatcaaaagggaaagacagcgcg

SEQ ID NO: 6 - protein, 11 aa, synthetic construct
  REGION 1..11: note = Lyn11
GCIKSKGKDSA

SEQ ID NO: 7 - DNA, 24 bp, synthetic construct
  misc_feature 1..24: note = Flexible linker
  source 1..24: mol_type = other DNA
ggagcagatagtgctggtagtgct

SEQ ID NO: 8 - protein, 8 aa, synthetic construct
  REGION 1..8: note = Flexible linker
GADSAGSA

SEQ ID NO: 9 - DNA, 54 bp, synthetic construct
  misc_feature 1..54: note = Nuclear exclusion signal (NES)
  source 1..54: mol_type = other DNA
aacctggtggacctccaaaagaagctggaggagctggagctggacgagcagcag

SEQ ID NO: 10 - protein, 18 aa, synthetic construct
  REGION 1..18: note = NES
NLVDLQKKLEELELDEQQ

SEQ ID NO: 11 - DNA, 945 bp, synthetic construct
  misc_feature 1..945: note = ERT2
  source 1..945: mol_type = other DNA
gctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgac

SEQ ID NO: 12 - protein, 315 aa, synthetic construct
  REGION 1..315: note = ERT2
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVD

SEQ ID NO: 13 - DNA, 36 bp, synthetic construct
  misc_feature 1..36: note = DEVD (PARP)
  source 1..36: mol_type = other DNA
aagaggaagggcgacgaggtggacggcgtggacgag

SEQ ID NO: 14 - protein, 12 aa, synthetic construct
  REGION 1..12: note = DEVD (PARP)
KRKGDEVDGVDE

SEQ ID NO: 15 - DNA, 12 bp, synthetic construct
  misc_feature 1..12: note = DEVD nt
  source 1..12: mol_type = other DNA
gacgaggtggac

SEQ ID NO: 16 - protein, 4 aa, synthetic construct
  REGION 1..4: note = DEVD AA
DEVD

SEQ ID NO: 17 - DNA, 741 bp, synthetic construct
  misc_feature 1..741: note = reverse tetracycline transactivator
  source 1..741: mol_type = other DNA
tctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggg

SEQ ID NO: 18 - protein, 247 aa, synthetic construct
  REGION 1..247: note = reverse tetracycline transactivator
SRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 19 - DNA, 63 bp, synthetic construct
  misc_feature 1..63: note = 3x Flag
  source 1..63: mol_type = other DNA
gactacaaagaccatgacggtgattataaagatcatgacatcgattacaaggatgacgatgac

SEQ ID NO: 20 - protein, 21 aa, synthetic construct
  REGION 1..21: note = 3X Flag
DYKDHDGDYKDHDIDYKDDDD

SEQ ID NO: 21 - DNA, 36 bp, synthetic construct
  misc_feature 1..36: note = DEVD (PARP) degenerate sequence
  source 1..36: mol_type = other DNA
aaacgcaaaggtgatgaagtagatggagtagacgaa

SEQ ID NO: 22 - protein, 12 aa, synthetic construct
  REGION 1..12: note = DEVD (PARP)
KRKGDEVDGVDE

SEQ ID NO: 23 - DNA, 945 bp, synthetic construct
  misc_feature 1..945: note = ERT2 (degenerate)
  source 1..945: mol_type = other DNA
gcaggggatatgcgcgcagcaaacctctggccttcacccctgatgataaagaggtcaaagaaaaactctctcgctctgtcactcaccgcagatcaaatggtgtccgcactgctggacgccgagcctccaatattgtacagtgaatacgatcccactcggcctttttccgaggcctccatgatggggctcctcacaaatctcgccgatcgagaacttgtgcatatgataaattgggccaaacgagtccctggtttcgttgatcttactctgcacgaccaagtacatctgctggagtgcgcttggctggaaattctcatgatcgggctggtatggaggagcatggaacatcccgtcaaactgctcttcgcccccaacctcttgctcgaccgaaatcaggggaagtgcgtggaagggatggtagaaatatttgatatgctcctggccacaagcagcagattcagaatgatgaacctccaaggcgaagaattcgtttgtttgaagagcatcatcctgctcaacagcggcgtctataccttcctctcctccacgttgaaaagccttgaggaaaaagatcacatccatagggtacttgataaaattactgataccctcatacaccttatggcaaaagcgggactcacacttcaacaacaacatcaaagactcgcacaattgttgttgattttgagccatataaggcatatgtcaaataagggaatggaacacctctactctatgaaatgtaagaatgtcgtcccactgtacgatcttctccttgaagctgcagatgctcatcggctccacgcacctacctctcggggcggcgctagcgttgaagagactgatcagtcacatctcgcgacagctggaagcacaagcagccacagcctccaaaaatactatattacaggcgaggccgaaggcttccccgctacggccgtggat

SEQ ID NO: 24 - protein, 315 aa, synthetic construct; identical to SEQ ID NO: 12
  REGION 1..315: note = ERT2

SEQ ID NO: 25 - DNA, 54 bp, synthetic construct
  misc_feature 1..54: note = NES (degenerate)
  source 1..54: mol_type = other DNA
aatcttgtagatcttcagaaaaaactcgaagaacttgagcttgatgaacaacaa

SEQ ID NO: 26 - protein, 18 aa, synthetic construct
  REGION 1..18: note = NES
NLVDLQKKLEELELDEQQ

SEQ ID NO: 27 - DNA, 1836 bp, synthetic construct
  misc_feature 1..1836: note = Lyn11-NES-ERT2-DEVD-rtTA NT (with flexible linker following Lyn11)
  source 1..1836: mol_type = other DNA
ggatgtataaaatcaaaagggaaagacagcgcgggagcagatagtgctggtagtgctggtaacctggtggacctccaaaagaagctggaggagctggagctggacgagcagcaggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaagaggaagggcgacgaggtggacggcgtggacgagtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggg

SEQ ID NO: 28 - protein, 612 aa, synthetic construct
  REGION 1..612: note = Lyn11-NES-ERT2-DEVD-rtTA A (with flexible linker following Lyn11)
GCIKSKGKDSAGADSAGSAGNLVDLQKKLEELELDEQQAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVDKRKGDEVDGVDESRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 29 - DNA, 891 bp, synthetic construct
  misc_feature 1..891: note = Lyn11-NES- DEVD-rtTA
  source 1..891: mol_type = other DNA
ggatgtataaaatcaaaagggaaagacagcgcgggagcagatagtgctggtagtgctggtaacctggtggacctccaaaagaagctggaggagctggagctggacgagcagcagaagaggaagggcgacgaggtggacggcgtggacgagtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggg

SEQ ID NO: 30 - protein, 297 aa, synthetic construct
  REGION 1..297: note = Lyn11-NES- DEVD-rtTA
GCIKSKGKDSAGADSAGSAGNLVDLQKKLEELELDEQQKRKGDEVDGVDESRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 31 - DNA, 1899 bp, synthetic construct
  misc_feature 1..1899: note = Lyn11-NES-ERT2-DEVD-rtTA-3xFLAG
  source 1..1899: mol_type = other DNA
ggatgtataaaatcaaaagggaaagacagcgcgggagcagatagtgctggtagtgctggtaacctggtggacctccaaaagaagctggaggagctggagctggacgagcagcaggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaagaggaagggcgacgaggtggacggcgtggacgagtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggggactacaaagaccatgacggtgattataaagatcatgacatcgattacaaggatgacgatgac

SEQ ID NO: 32 - protein, 633 aa, synthetic construct
  REGION 1..633: note = Lyn11-NES-ERT2-DEVD-rtTA-3xFLAG
GCIKSKGKDSAGADSAGSAGNLVDLQKKLEELELDEQQAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVDKRKGDEVDGVDESRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPGDYKDHDGDYKDHDIDYKDDDD

SEQ ID NO: 33 - DNA, 954 bp, synthetic construct
  misc_feature 1..954: note = Lyn11-NES- DEVD-rtTA-3xFLAG
  source 1..954: mol_type = other DNA
ggatgtataaaatcaaaagggaaagacagcgcgggagcagatagtgctggtagtgctggtaacctggtggacctccaaaagaagctggaggagctggagctggacgagcagcagaagaggaagggcgacgaggtggacggcgtggacgagtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggggactacaaagaccatgacggtgattataaagatcatgacatcgattacaaggatgacgatgac

SEQ ID NO: 34 - protein, 318 aa, synthetic construct
  REGION 1..318: note = Lyn11-NES- DEVD-rtTA-3xFLAG
GCIKSKGKDSAGADSAGSAGNLVDLQKKLEELELDEQQKRKGDEVDGVDESRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPGDYKDHDGDYKDHDIDYKDDDD

SEQ ID NO: 35 - DNA, 2769 bp, synthetic construct
  misc_feature 1..2769: note = ERT2-DEVD-rtTA-3xFLAG-DEVD-ERT2 NT with GCA after flag
  source 1..2769: mol_type = other DNA
gctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaagaggaagggcgacgaggtggacggcgtggacgagtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggggactacaaagaccatgacggtgattataaagatcatgacatcgattacaaggatgacgatgacgcaaaacgcaaaggtgatgaagtagatggagtagacgaagcaggggatatgcgcgcagcaaacctctggccttcacccctgatgataaagaggtcaaagaaaaactctctcgctctgtcactcaccgcagatcaaatggtgtccgcactgctggacgccgagcctccaatattgtacagtgaatacgatcccactcggcctttttccgaggcctccatgatggggctcctcacaaatctcgccgatcgagaacttgtgcatatgataaattgggccaaacgagtccctggtttcgttgatcttactctgcacgaccaagtacatctgctggagtgcgcttggctggaaattctcatgatcgggctggtatggaggagcatggaacatcccgtcaaactgctcttcgcccccaacctcttgctcgaccgaaatcaggggaagtgcgtggaagggatggtagaaatatttgatatgctcctggccacaagcagcagattcagaatgatgaacctccaaggcgaagaattcgtttgtttgaagagcatcatcctgctcaacagcggcgtctataccttcctctcctccacgttgaaaagccttgaggaaaaagatcacatccatagggtacttgataaaattactgataccctcatacaccttatggcaaaagcgggactcacacttcaacaacaacatcaaagactcgcacaattgttgttgattttgagccatataaggcatatgtcaaataagggaatggaacacctctactctatgaaatgtaagaatgtcgtcccactgtacgatcttctccttgaagctgcagatgctcatcggctccacgcacctacctctcggggcggcgctagcgttgaagagactgatcagtcacatctcgcgacagctggaagcacaagcagccacagcctccaaaaatactatattacaggcgaggccgaaggcttccccgctacggccgtggat

SEQ ID NO: 36 - protein, 923 aa, synthetic construct
  REGION 1..923: note = ERT2-DEVD-rtTA-3xFLAG-DEVD-ERT2
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVDKRKGDEVDGVDESRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPGDYKDHDGDYKDHDIDYKDDDDAKRKGDEVDGVDEAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVD

SEQ ID NO: 37 - DNA, 2766 bp, synthetic construct
  misc_feature 1..2766: note = ERT2-DEVD-rtTA-3xFLAG-DEVD-ERT2 NT w/out GCA after flag
  source 1..2766: mol_type = other DNA
gctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaagaggaagggcgacgaggtggacggcgtggacgagtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggggactacaaagaccatgacggtgattataaagatcatgacatcgattacaaggatgacgatgacaaacgcaaaggtgatgaagtagatggagtagacgaagcaggggatatgcgcgcagcaaacctctggccttcacccctgatgataaagaggtcaaagaaaaactctctcgctctgtcactcaccgcagatcaaatggtgtccgcactgctggacgccgagcctccaatattgtacagtgaatacgatcccactcggcctttttccgaggcctccatgatggggctcctcacaaatctcgccgatcgagaacttgtgcatatgataaattgggccaaacgagtccctggtttcgttgatcttactctgcacgaccaagtacatctgctggagtgcgcttggctggaaattctcatgatcgggctggtatggaggagcatggaacatcccgtcaaactgctcttcgcccccaacctcttgctcgaccgaaatcaggggaagtgcgtggaagggatggtagaaatatttgatatgctcctggccacaagcagcagattcagaatgatgaacctccaaggcgaagaattcgtttgtttgaagagcatcatcctgctcaacagcggcgtctataccttcctctcctccacgttgaaaagccttgaggaaaaagatcacatccatagggtacttgataaaattactgataccctcatacaccttatggcaaaagcgggactcacacttcaacaacaacatcaaagactcgcacaattgttgttgattttgagccatataaggcatatgtcaaataagggaatggaacacctctactctatgaaatgtaagaatgtcgtcccactgtacgatcttctccttgaagctgcagatgctcatcggctccacgcacctacctctcggggcggcgctagcgttgaagagactgatcagtcacatctcgcgacagctggaagcacaagcagccacagcctccaaaaatactatattacaggcgaggccgaaggcttccccgctacggccgtggat

SEQ ID NO: 38 - protein, 922 aa, synthetic construct
  REGION 1..922: note = ERT2-DEVD-rtTA-3xFLAG-DEVD-ERT2 NT w/out GCA after flag
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVDKRKGDEVDGVDESRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPGDYKDHDGDYKDHDIDYKDDDDKRKGDEVDGVDEAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATAVD

SEQ ID NO: 39 - DNA, 1503 bp, synthetic construct
  misc_feature 1..1503: note = MCD8-NES- DEVD-rtTA
  source 1..1503: mol_type = other DNA
atggcctcaccgttgacccgctttctgtcgctgaacctgctgctgctgggtgagtcgattatcctggggagtggagaagctaagccacaggcacccgaactccgaatctttccaaagaaaatggacgccgaacttggtcagaaggtggacctggtatgtgaagtgttggggtccgtttcgcaaggatgctcttggctcttccagaactccagctccaaactcccccagcccaccttcgttgtctatatggcttcatcccacaacaagataacgtgggacgagaagctgaattcgtcgaaactgttttctgccatgagggacacgaataataagtacgttctcaccctgaacaagttcagcaaggaaaacgaaggctactatttctgctcagtcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacagccgcggatctaacctggtggacctccaaaagaagctggaggagctggagctggacgagcagcagaagaggaagggcgacgaggtggacggcgtggacgagtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccggg

SEQ ID NO: 40 - protein, 501 aa, synthetic construct
  REGION 1..501: note = MCD8-NES- DEVD-rtTA
MASPLTRFLSLNLLLLGESIILGSGEAKPQAPELRIFPKKMDAELGQKVDLVCEVLGSVSQGCSWLFQNSSSKLPQPTFVVYMASSHNKITWDEKLNSSKLFSAMRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVALLLSLIITLICYHSRGSNLVDLQKKLEELELDEQQKRKGDEVDGVDESRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 41 - DNA, 204 bp, synthetic construct
  misc_feature 1..204: note = Mito-CAVP (N-terminal end of SMAC)
  source 1..204: mol_type = other DNA
atggcggctctgaagagttggctgtcgcccatcgtaacttcattcttcaggtacagacagtgtttgtgtgttcctgttgtggctaactttaagaagcggtgtttctcagaattgataagaccatggcacaaaactgtgacgattggctttggagtaaccctgtgtgcggttcctattgcacagaaatcagagcctcattccctt

SEQ ID NO: 42 - protein, 68 aa, synthetic construct
  REGION 1..68: note = Mito-CAVP (N-terminal end of SMAC)
MAALKSWLSRSVTSFFRYRQCLCVPVVANFKKRCFSELIRPWHKTVTIGFGVTLCAVPIAQAVYTLTS

SEQ ID NO: 43 - DNA, 705 bp, synthetic construct
  misc_feature 1..705: note = Gal4-VP16 transactivator
  source 1..705: mol_type = other DNA
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 44 - DNA, 1272 bp, synthetic construct
  misc_feature 1..1272: note = FLPo recombinase
  source 1..1272: mol_type = other DNA
atgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctaa

SEQ ID NO: 45 - DNA, 1041 bp, synthetic construct
  misc_feature 1..1041: note = Cre recobminase
  source 1..1041: mol_type = other DNA
atgaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatctcgagccatcttaa

SEQ ID NO: 46 - protein, 5 aa, synthetic construct
  VAR_SEQ 4: note = X means D or A
DQVXA

SEQ ID NO: 47 - protein, 4 aa, synthetic construct
RICG